(19) United States
(12) Patent Application Publication
(10) Pub. No.: US 2005/0136532 A1
(43) Pub. Date: Jun. 23, 2005

(54) System of scattering surface plasmon resonance for detecting a target

(75) Inventor: Pao-Hung Lin, Changhua City (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Chutung (TW)

(21) Appl. No.: 10/841,642

(22) Filed: May 10, 2004

(30) Foreign Application Priority Data
Jun. 11, 2003 (TW) 092115890

(51) Int. Cl. G01N 33/53; G01N 33/567; C12M 1/34

(52) U.S. Cl. 435/287.2; 356/128

(57) ABSTRACT

A system of scattering surface plasmon resonance for detecting a target is disclosed. The system of scattering surface plasmon resonance includes: a base slide having a metal layer and a plurality of insulating nano-particles thereon, wherein said plurality of insulating nano-particles are dispersed on the surface of said metal layer for binding or coupling at least one detecting probe capable of binding or hybridizing said target; a light source providing an incident light to said base slide for inducing or exciting a surface plasmon resonance (SPR) signal; and a detector for receiving said SPR signal. The disclosed system can improve the adherence between the traditional slides and the probe molecules, enhances the scattering to increase the sensitivity of SPW detection, and facilitate and simplify the sample preparation for SPR.

### BACKGROUND OF THE INVENTION

1. Field of the Invention

The present invention relates to technologies of surface plasmon resonance (SPR) and, more particularly, to systems of scattering surface plasmon resonance on a slide coated with insulating nanoparticles.

2. Description of Related Art

Currently, photo detection is widely applied for the signal detection of many biosensors. Among them, surface plasmon resonance (SPR) is famous in the field of monitoring of changes of environments, analysis of pharmaceutical effects, and diagnosis of diseases through binding of bio-molecules. For example, SPR is very useful for the determination of the interaction between antigens and antibodies, the interaction of pepetides or proteins, the biocompatibilities between bio-molecules, the hybridization of DNA/RNA fragments, or non-specific adsorption of proteins. In addition, the advantages of SPR such as high speed, high sensitivity, high specificity, high throughput for parallel screening, and exemption from pretreatment for labeling molecules also make SPR popular.

A conventional SPR system can be seen in FIG. 1. Basically, the SPR system includes a light source 1, a metal film 2, a target 3 for analysis, and a detector 4. As the light from light source 1 is introduced to the metal film 2, a surface plasma wave (SPW) 12 is induced from the metal film 2. The surface plasma wave (SPW) generates on the spots between the metal film and the charge of the dielectric layer of the target molecules. When the incident light 11 (with incident angle θ1) and the surface plasma wave meet the conditions of the resonance, all the energy of the incident light will transfer to the surface plasma wave. Then the distribution of the intensity of the reflecting light 13 with various reflecting angle varies very greatly. Only the reflection of specific reflecting angle θ2 is enhanced, and intensities of angles except specific angle decrease dramatically (i.e. SPR phenomenon). The specific reflecting angle is called resonance angle for SPR. The intensity of the reflection or the reflecting angle of SPR can be detected through a detector. Moreover, the resonance angle for SPR also depends on the refractive index of the molecules adjacent to or close to the metal film. In addition, the resonance angle for SPR also depends on the concentration, the species of medium, or the molecules adjacent to the metal film. Hence, SPR can be used for determining the concentration of target molecules on metal films without any labeling pretreatment. So far, the most popular way for detecting SPR signals is to measure the change of light intensities of reflecting light 13. However, interference such as diffraction often happens on the photo detector as the laser light of the light source is reflected. The diffraction can be improved when the laser is replaced with an incandescent lamp. Unfortunately, the shadow of the incandescent lamp becomes another source of interference. For improving the diffraction interference, the photo detector is arranged above the target molecules as the SPR is measured. However, only one spot without definite SPR is observed. In other words, no reliable SPR signals can be detected as the photo detector is arranged above the target molecules.

In addition, metal films are required for the conventional SPR for binding the target molecules for analysis. It is difficult for the generated surface plasma wave (SPW) to be scattered to the target molecules since there is almost no scattering spot on the smooth metal surface. Moreover, since the binding technologies for the metal film and the target molecules are not easy to be achieved, the intention to use SPR for analysis is discouraged. Therefore, improvement for binding the target molecules (e.g. bio-molecules) and the metal films and substantial reduction for interference become very important.

Therefore, it is desirable to provide an improved system of scattering surface plasma resonance for detecting a target to mitigate and/or obviate the aforementioned problems.

### SUMMARY OF THE INVENTION

The object of the present invention is to provide a system of scattering surface plasma resonance for detecting a target, to improve the adherence of the probe molecules or the target molecules (e.g. bio-molecules) on the slides for carrying probe molecules or target molecules through modification of the surface of the slides, to increase the throughout for SPR analysis, and to facilitate the procedures for using SPR.

Another object of the present invention is to provide a detecting-slide having a coat of nano-particles for a surface plasmon resonance (SPR) system, to improve the adherence of the probe molecules or the target molecules (e.g. bio-molecules) on the slides for carrying probe molecules or target molecules through modification of the surface of the slides, to increase the throughout for SPR analysis, and to facilitate the procedures for using SPR.

To achieve the object, the system of scattering surface plasma resonance for detecting a target of the present invention includes: a base slide having a metal layer and a plurality of insulating nano-particles thereon, wherein said insulating nano-particles are dispersed on the surface of said metal layer for coupling at least one detecting probe capable of binding or hybridizing said target; a light source providing an incident light to said base slide for inducing or exciting a surface plasmon resonance (SPR) signal, wherein said SPR signal is scattered by said insulating nano-particles, said detecting probe and said target; and a detector for receiving said SPR signal scattered by said base slide having detecting probe and optionally said target.

To achieve the other object, the detecting-slide having a coat of nano-particles for a surface plasmon resonance (SPR) system of the present invention includes: a slide; a metal film formed on said slide; and a plurality of insulating nano-particles, which is dispersed on said metal film for coupling with at least one detecting probe capable of binding or hybridizing said target; wherein a SPR signal induced or excited by said SPR system is scattered by said insulating nano-particles onto said detecting probes and said target.

The insulating nano-particles can be any conventional insulating nano-particles. Preferably, the insulating nano-particles are made of oxides. More preferably, the insulating nano-particles comprise 45 parts by weight to 100 parts by weight of silica, alumina, antimony oxide, zinc oxide, chromium oxide or mixtures thereof. Generally, the insulating nano-particles function as the transferring background of micro-developing images for achieving high flux detection. The light source of the present invention can be any conventional light source. Preferably, the light source of the present invention is a laser. The detector of the present invention can be any conventional photo detector. Preferably, the detector of the present invention is a charge couple device (CCD). The target or the probe for analysis by the detection system of the present invention can be any conventional molecule or bio-molecule. Preferably, the targets or probes for analysis by the detection system of the present invention are antigens, antibodies, protein molecules, DNA fragments, RNA fragments or a combination thereof.

Other objects, advantages, and novel features of the invention will become more apparent from the following detailed description when taken in conjunction with the accompanying drawings.

### BRIEF DESCRIPTION OF THE DRAWINGS

FIG. 1 is a diagram of the conventional SPR of prior arts.

FIG. 2 is a diagram of the configuration of the SPR of an embodiment of the present invention.

FIG. 3 is a diagram of the base slide used in the SPR of an embodiment of the present invention.

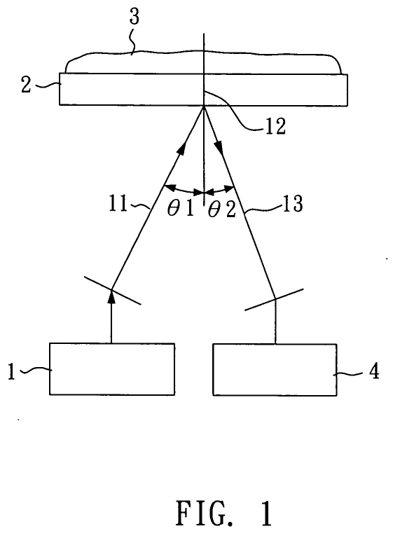

### DETAILED DESCRIPTION OF THE PREFERRED EMBODIMENT

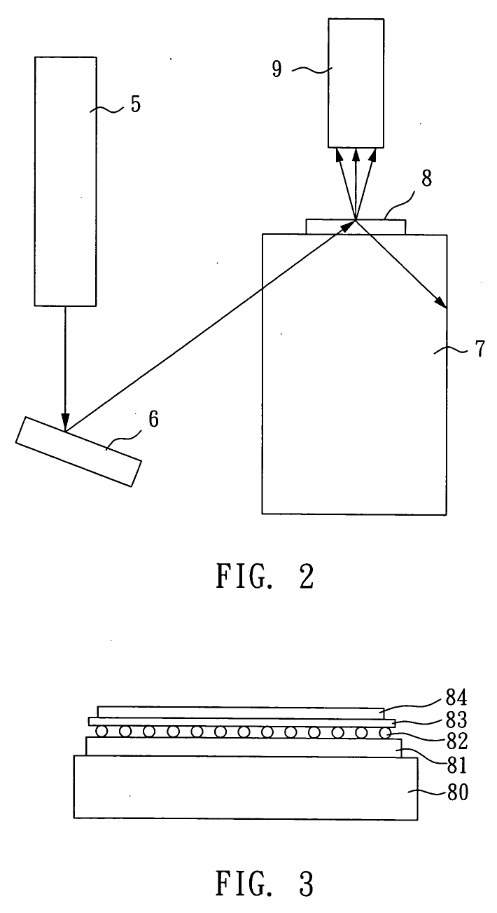

With reference to FIG. 2, there is shown an embodiment of a system of scattering surface plasma resonance of the present invention. The system of scattering surface plasma resonance of the present invention includes a light source 5, a reflecting mirror 6, a prism 7, a base slide 8, and a detector 9. The detector can be arranged in a position of the path of the reflecting light passing through the prism 7 (for SPR signals) or in a position on the top of the slide 8 and the prism 7 if the surface plasmon resonance fluorescence (SPRF) is measured. In FIG. 2, the detector is arranged on the top of the slide 8 and the prism 7. The base slide 8 of the present embodiment includes a glass 80, a metal film 81, and a plurality of insulating nanoparticles 82 disbursed on the metal film 81 (see FIG. 3). As shown in FIG. 3, the metal film 81 is coated on the glass 80. The insulating nanoparticles of the present invention can be any insulating nanoparticles without electrical conductivity. The insulating nanoparticles of the present embodiment are 45 parts by weight to 100 parts by weight of silica, alumina, antimony oxide, zinc oxide, or chromium oxide. Since the surface of the insulating nanoparticles is similar to that of conventional glass, therefore, the probe molecules or the target molecules (or the bio-molecules) are easy to be attached, coupled or bound on the surface of the insulating nanoparticles. Therefore, the surface of the traditional metal surface can be modified for the easy adherence for probe molecules or target molecules. The modified surface of the combined layers of insulating nanoparticles and the metal film can facilitate the conjugation of the probe molecules or the target molecules and the surface of the slides.

The laser light from the light source 5 is reflected by the reflecting mirror 6 and is projected through the prism 7 and the base slide 8. The arrangement of the reflecting mirror 6 and the prism 7 depends on the practical needs for measurement (e.g. SPR or SPRF). The metal film induces SPR signals and generates a reflecting light in the glass 80 (SPR phenomenon).

Since the wavelength of SPW is around 200 nm which is bigger than the size of the insulating nanoparticles (around 100 nm), the SPW can scattered to the probe molecules 83 and the target molecules 84 by using the insulating nanoparticles as a background for scattering (Rayleigh Scattering). According to the rules for elastic scattering, the wavelength of the scattered light is almost the same as that of the light before scattering. Therefore, by detecting the SPR signals received by the detector 9, the binding or the hybridization between the probes and the targets can be detected.

The system of the present invention improves the adherence between the traditional slides and the probe molecules (or the target molecules), and enhances the scattering to increase the sensitivity of SPW detection by using nanoparticles as the scattering background. Therefore, the sample preparation for SPR can be facilitated and simplified, and the throughput for SPR analysis can be increased.

Although the present invention has been explained in relation to its preferred embodiment, it is to be understood that many other possible modifications and variations can be made without departing from the spirit and scope of the invention as hereinafter claimed.

1. A system of scattering surface plasmon resonance for detecting a target, comprising:
a base slide having a metal layer and a plurality of insulating nano-particles thereon, wherein said plurality of insulating nano-particles are dispersed on the surface of said metal layer for binding or coupling at least one detecting probe capable of binding or hybridizing said target;
a light source providing an incident light to said base slide for inducing or exciting a surface plasmon resonance (SPR) signal, wherein said SPR signal is scattered by said insulating nano-particles, said detecting probe and said target; and
a detector for receiving said SPR signal scattered by said base slide having the at least one detecting probe and optionally said target.

2. The system as claimed in claim 1, wherein said insulating nano-particles are made of oxides.

3. The system as claimed in claim 2, wherein said insulating nano-particles comprising 45 part by weight to 100 part by weight of silica, alumina, antimony oxide, zinc oxide, chromium oxide or mixtures thereof.

4. The system as claimed in claim 1, wherein said insulating nano-particles function as the transferring background of micro-developing images for achieving high flux detection.

5. The system as claimed in claim 1, wherein said light source is a laser.

6. The system as claimed in claim 1, wherein said detector is a charge couple device (CCD).

7. The system as claimed in claim 1, wherein said probe or said target is selected from the group consisting of antigens, antibodies, protein molecules, DNA fragments, and RNA fragments.

8. A detecting-slide having a coat of nano-particles for a surface plasmon resonance (SPR) system, comprising:
a slide;
a metal film formed on said slide; and
a plurality of insulating nano-particles, which is dispersed on said metal film for coupling with at least one detecting probe capable of binding or hybridizing said target;
wherein an SPR signal induced or excited by said SPR system is scattered by said insulating nano-particles onto said detecting probes and said target.

9. The detecting-slide as claimed in claim 8, wherein said insulating nano-particles are made of oxides.

10. The detection-slide as claimed in claim 9, wherein said insulating nano-particles having 45 part by weight to 100 part by weight of silica, alumina, antimony oxide, zinc oxide, or chromium oxide.

11. The detection-slide as claimed in claim 8, wherein said insulating nano-particles function as the transferring background of micro-developing images for achieving high flux detection.

12. The detection-slide as claimed in claim 8, wherein said detecting probes or said target are selected from the group consisting of antigens, antibodies, protein molecules, DNA fragments, and RNA fragments.
